(19) United States
(12) Patent Application Publication
(10) Pub. No.: US 2011/0076230 A1
(43) Pub. Date: Mar. 31, 2011

(54) Novel Substituted Indoles

(76) Inventors: James C. Barrow, Arnold, MD (US); Scott Harrison, Elkins Park, PA (US); James Mulhearn, Saint Davids, PA (US); Scott Wolkenberg, Jenkintown, PA (US)

(21) Appl. No.: 12/995,353

(22) PCT Filed: May 28, 2009
(86) PCT No.: PCT/US09/45403

§ 371 Date: Nov. 30, 2010

Related U.S. Application Data
(60) Provisional application No. 61/130,449, filed on May 30, 2008.

(51) Int. Cl. A61K 51/00; A61K 49/00; A61K 49/06; A61K 31/4439; A61K 31/437; C07D 471/04; C07D 401/04; C07D 401/14; A61K 31/497; A61P 25/28; A61P 25/18

(52) U.S. Cl. 424/1.11; 424/9.1; 424/9.3; 514/339; 514/300; 546/113; 546/272.4; 546/274.1; 546/277.4; 544/364; 514/253.09

(57) ABSTRACT

The present invention relates to novel amyloid binding compounds and methods for measuring effects of the compounds, by measuring changes of amyloid plaque level in living patients. More specifically, the present invention relates to a method of using the compounds of this invention as tracers in positron emission tomography (PET) imaging to study amyloid deposits in brain in vivo to allow diagnosis of Alzheimer's disease. Thus, the present invention relates to use of the novel amyloid binding compounds as a diagnostic. The invention further relates to a method of measuring clinical efficacy of Alzheimer's disease therapeutic agents. Specifically, the present invention relates to novel aryl or heteroaryl substituted indole derivatives, compositions, and therapeutic uses and processes for making such compounds.

### FIELD OF THE INVENTION

The present invention relates to novel aryl or heteroaryl substituted indole derivatives, compositions, and therapeutic uses and processes for making such compounds. The invention is further directed to ²H, ³H, ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁶Br, ⁷⁷Br, ¹²³I, ¹²⁴I and ¹³¹I isotopically labeled aryl or heteroaryl substituted indole derivative compounds. In particular, the present invention is directed to ¹¹C, ¹³C, ¹⁴C, ¹⁸F, ¹⁵O, ¹³N, ³⁵S, ²H, and ³H isotopes of aryl or heteroaryl substituted indole and methods of their preparation.

The invention also relates to novel aryl or heteroaryl substituted indole derivatives which are suitable for imaging amyloid deposits in living patients. More specifically, the present invention relates to a method of using the compounds of this invention as tracers in positron emission tomography (PET) imaging to study amyloid deposits in brain in vivo to allow diagnosis of Alzheimer's disease. The invention further relates to a method of measuring clinical efficacy of Alzheimer's disease therapeutic agents.

### BACKGROUND OF THE INVENTION

Noninvasive nuclear imaging techniques can be used to obtain basic and diagnostic information about the physiology and biochemistry of a variety of living subjects including experimental animals, normal humans and patients. These techniques rely on the use of sophisticated imaging instrumentation that is capable of detecting radiation emitted from radiotracers administered to such living subjects. The information obtained can be reconstructed to provide planar and tomographic images that reveal distribution of the radiotracer as a function of time. Use of appropriately designed radiotracers can result in images which contain information on the structure, function and most importantly, the physiology and biochemistry of the subject. Much of this information cannot be obtained by other means. The radiotracers used in these studies are designed to have defined behaviors in vivo which permit the determination of specific information concerning the physiology or biochemistry of the subject or the effects that various diseases or drugs have on the physiology or biochemistry of the subject. Currently, radiotracers are available for obtaining useful information concerning such things as cardiac function, myocardial blood flow, lung perfusion, liver function, brain blood flow, regional brain glucose and oxygen metabolism.

For noninvasive in vivo imaging, compounds can be labeled with either positron- or gamma-emitting radionuclides. The most commonly used positron emitting (PET) radionuclides are ¹¹C, ¹⁸F, ¹⁵O and ¹³N, all of which are accelerator produced, and have half-lives of 20, 110, 2 and 10 minutes, respectively. Since the half-lives of these radionuclides are so short, it is only feasible to use them at institutions that have an accelerator on site or very close by for their production, thus limiting their use. Several gamma emitting radiotracers are available which can be used by essentially any hospital in the U.S. and most hospitals worldwide. The most widely used of these are ⁹⁹Tc, ²⁰¹Tl and ¹²³I.

In a typical PET study, a small amount of radiotracer is administered to the experimental animal, normal human or patient being tested. The radiotracer then circulates in the blood of the subject and may be absorbed in certain tissues. The radiotracer may be preferentially retained in some of these tissues because of specific enzymatic conversion or by specific binding to macromolecular structures such as proteins. Using sophisticated imaging instrumentation to detect positron emission, the amount of radiotracer is then non-invasively assessed in the various tissues in the body. The resulting data are analyzed to provide quantitative spatial information of the in vivo biological process for which the tracer was designed. PET gives pharmaceutical research investigators the capability to assess biochemical changes or metabolic effects of a drug candidate in vivo for extended periods of time, and PET can be used to measure drug distribution, thus allowing the evaluation of the pharmacokinetics and pharmacodynamics of a particular drug candidate under study. Importantly, PET tracers can be designed and used to quantitate the presence of binding sites in tissues. Consequently, interest in PET tracers for drug development has been expanding based on the development of isotopically labeled biochemicals and appropriate detection devices to detect the radioactivity by external imaging.

Noninvasive nuclear imaging techniques such as PET have been particularly important in providing the ability to study neurological diseases and disorders, including stroke, Parkinson's disease, epilepsy, cerebral tumors and Alzheimer's disease.

Alzheimer's disease is the most common form of dementia. It is a neurologic disease characterized by loss of mental ability severe enough to interfere with normal activities of daily living. It usually occurs in old age, and is marked by a decline in cognitive functions such as remembering, reasoning, and planning. All forms of Alzheimer's disease pathology are characterized by the accumulation of amyloid Aβ-peptide. See Cai, L. et al., Current Medicinal Chemistry, 2007, 14, 19-52; Chandra, R. et al. J. Med. Chem. 2007, 50, 2415-2423; Qu, W. et al., J. Med. Chem. 2007, 50, 3380-3387; Cai, L. et al., J. Med. Chem. 2007, 50, 4746-4758; and Qu, W. et al., J. Med. Chem. 2007, 50, 2157-2165. PET and single photon emission computed tomography (SPECT), are effective in monitoring the accumulation of amyloid deposits in the brain and correlating it to the progression of AD (Shoghi-Jadid et al. The American Journal of Geriatric Psychiatry 2002, 10, 24; Miller, Science, 2006, 313, 1376; Coimbra et al. Curr. Top. Med. Chem. 2006, 6, 629; Nordberg, Lancet Neurol. 2004, 3, 519). Thus, there is a need for non-toxic amyloid binding radiotracers that can rapidly cross the blood-brain barrier, that have potent, specific binding properties and low non-specific binding properties, that can be used in diagnostics, and that can rapidly clear from the system. These compounds also can be used in monitoring the effectiveness of treatment programs given to Alzheimer's patients by measuring the changes of amyloid plaque level. See Coimbra et al. Curr. Top. Med. Chem. 2006, 6, 629); Mathis et al. J. Med. Chem. 2003, 46, 2740; Klunk et al. Ann Neurol. 2004, 55, 306 for background discussion on properties of amyloid binding. See WO 2007/086800, WO2007149030, WO 2007/002540, WO 2007/074786, WO 2002/016333, WO2003048137, WO2002085903, and WO 2004/083195 for examples of compounds and methods used in the treatment of Alzheimer's disease. See also U.S. Pat. No. 6,696,039, US2004/0131545, U.S. Pat. No. 6,001,331, WO2004/032975, WO2004/064869, US2005/0043377, WO2007/033080, U.S. Pat. No. 4,038,396, WO2006044503, WO2006044503, WO2007070173, and U.S. Pat. No. 3,899,506.

While the primary use of the isotopically labeled compounds of this invention is in positron emission tomography, which is an in vivo analysis technique, certain of the isotopically labeled compounds can be used for methods other than PET analyses. In particular, ¹⁴C and ³H labeled compounds can be used in in vitro and in viva methods for the determination of binding, receptor occupancy and metabolic studies including covalent labeling. In particular, various isotopically labeled compounds find utility in magnetic resonance imaging, autoradiography and other similar analytical tools.

### SUMMARY OF THE INVENTION

The present invention relates to novel amyloid binding compounds and methods for measuring effects of the compounds, by measuring changes of amyloid plaque level in living patients. More specifically, the present invention relates to a method of using the compounds of this invention as tracers in positron emission tomography (PET) imaging to study amyloid deposits in brain in viva to allow diagnosis of Alzheimer's disease. Thus, the present invention relates to use of the novel amyloid binding compounds as a diagnostic. The invention further relates to a method of measuring clinical efficacy of Alzheimer's disease therapeutic agents. Specifically, the present invention relates to novel aryl or heteroaryl substituted indole derivatives, compositions, and therapeutic uses and processes for making such compounds. The invention is further directed to ²H, ³H, ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁶Br, ⁷⁷Br, ¹²³I, ¹²⁴I and ¹³¹I isotopically labeled aryl or heteroaryl substituted indole derivative compounds, compositions, methods of their preparation and their use as PET tracers in diagnosing and measuring the effects of a compound in the treatment of Alzheimer's Disease. The present invention also relates to non-toxic amyloid binding compounds that can rapidly cross the blood brain barrier, have low non-specific binding properties and rapidly clear from the system. This and other aspects of the invention will be realized upon review of the specification in its entirety.

### DETAILED DESCRIPTION OF THE INVENTION

In one aspect of the invention, there is provided a compound according to formula I:

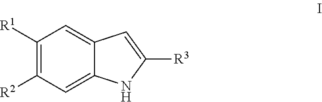

or a pharmaceutically acceptable salt, solvate or in vivo hydrolysable ester thereof, wherein:
R³ is pyridyl optionally substituted with 1 to 3 groups of R⁴, R⁵, or R⁶, with the proviso that when two of R⁴, R⁵ and R⁶, is hydrogen and the remainder of R⁴, R⁵ or R⁶ is N(R²)₂, piperazinyl or methyl piperazinyl, and one of R¹ and R² is hydrogen then the other of R¹ and R² is not methoxy or halogen;
R represents hydrogen, or —C₁₋₆alkyl, said alkyl optionally substituted with halo;
R¹, R², R⁴, R⁵, and R⁶ independently represent hydrogen, —C₅₋₁₀ aryl, —C₅₋₁₀ heterocyclyl, —N(R²)₂, CN, —(CH₂)ₙhalo, CF₃, —O(CH₂)ₙR, —O(CH₂)ₙC₅₋₁₀ heterocyclyl, —C₁₋₆alkyl, —OCF₃, —O(CH₂)ₛF, —(O(CH2)ₛ)ₚ(CH₂)ₛhalo, —(O(CH₂)ₛ)ₚOR, said alkyl, aryl, and heterocyclyl optionally substituted with 1 to 3 groups of R^{a},
or when two of R⁴, R⁵ and R6 are adjacent to each other on the R³ pyridyl then they may combine with the atoms to which they are attached to form a 9-10 membered heterocyclic ring optionally interrupted by NR, O, or S, said heterocyclyl optionally substituted with 1 to 3 groups of R^{a};
R^{a} represents —CN, NO₂, halo, CF₃, —C₁₋₆alkyl, —C₁₋₆alkenyl, —C₁₋₆alkynyl, —(CH₂)ₙhalo, —OR, —NRR¹, —C(═NR¹)NR²R⁵, —NR¹COR², —NR¹CO₂R², —NR¹SO₂R⁵, —NR¹CONR²R⁵, —SR⁵, —SOR⁵, —SO₂R⁵, —SO₂NR¹R², —COR¹, —CO₂R¹, —CONR¹R², —C(═NR¹)R², or —C(═NOR¹)R²;
n represents 0-6;
s represents 1-6; and
p represents 1-4.

One aspect of this invention is realized when R³ is pyridyl optionally substituted with 1 to 3 groups of R⁴, R⁵, and R⁶ independently selected from hydrogen, —C₅₋₁₀ heterocyclyl, —N(R²)₂, —(CH₂)ₙhalo, —O(CH₂)ₙR, —C₁₋₆alkyl, —OCF₃, —O(CH₂)ₛF, said alkyl, aryl, and heterocyclyl optionally substituted with 1 to 3 groups of R^{a}.

One aspect of this invention is realized when R³ is substituted pyridyl. A sub-embodiment of this invention is realized when R³ is pyridyl substituted with halo, methylamine, piperazinyl, triazolyl, imidazolyl, or pyrazolyl and all other variables are as originally described. A sub-embodiment of this invention is realized when R³ is pyridyl substituted with halo, preferably fluorine. Another sub-embodiment of this invention is realized when R³ is pyridyl substituted with triazolyl. Another sub-embodiment of this invention is realized when R³ is pyridyl substituted with imidazolyl. Still another sub-embodiment of this invention is realized when R³ is pyridyl substituted with pyrazolyl.

Another aspect of this invention is realized when two of R⁴, R⁵ and R⁶ adjacent to each other on the R³ pyridyl combine with the atoms to which they are attached to form a 9-10 membered heterocyclic ring, including fused rings, optionally interrupted by NR, O, or S, said heterocyclic ring optionally substituted with R^{a}. A sub-embodiment of this invention is realized by structural formula II:

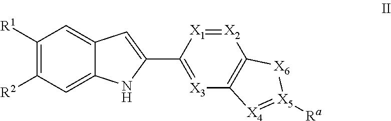

or a pharmaceutically acceptable salt, solvate or in vivo hydrolysable ester thereof, wherein: X₁-X₅ are N or CH, provided only one of X₁-X₃ is N at any given time; and X₆ is NR, —O—, CH₂ or S and all other variables are as previously described. Another sub-embodiment of this invention is realized when X₁ through X₆ are added to form a pyrrolo pyridinyl and all other variables are as previously described.

Another aspect of this invention is realized when R¹ and R² on the indolyl are selected from the group consisting of hydrogen, CN, —(CH₂)ₙhalo, —O(CH₂)ₙR, —O(CH₂)ₙhalo, —O(CH₂)ₙC₅₋₁₀ heterocyclyl, O(CH₂)ₙC₆₋₁₀ aryl or —C₁₋₆alkyl. A sub-embodiment of this invention is realized when one of R¹ and R² is hydrogen and the other is O(CH₂)ₙF, F, Br, Cl, CN, methoxy, methyl, hydroxyl, benzyloxy.

Yet another aspect of this invention is realized when R^{a} represents halo, —CN, NO₂, —C₁₋₆alkyl,—OR, —N(R)₂, —NRCOR², —NRCO₂R, or —C₅₋₁₀ heterocyclyl.

Another aspect of the invention is realized when the compounds of formulas I and II are ²H, ³H, ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁶Br, ⁷⁷Br, ¹²³I, ¹²⁴I and ¹³¹I isotopically labeled.

Examples of compounds of this invention are:

| Structure | Nomenclature | M + 1 |
|---|---|---|
| 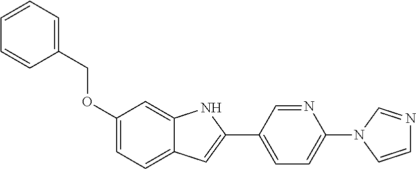 | 6-(benzyloxy)-2-[6-(1H- imidazol-1-yl)pyridin-3-yl]- 1H-indole | 367 |
| 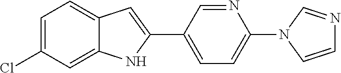 | 6-chloro-2-[6-(1H-imidazol-1- yl)pyridin-3-yl]-1H-indole | 295 |
| 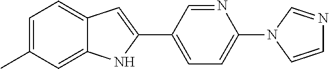 | 2-[6-(1H-imidazol-1- yl)pyridin-3-yl]-6-methyl-1H- indole | 275 |
| 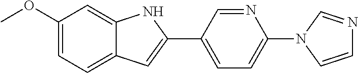 | 2-[6-(1H-imidazol-1- yl)pyridin-3-yl]-6-methoxy- 1H-indole | 291 |
| 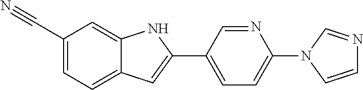 | 2-[6-(1H-imidazol-1- yl)pyridin-3-yl]-1H-indole-6- carbonitrile | 286 |
| 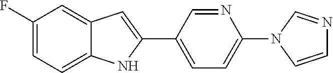 | 5-fluoro-2-[6-(1H-imidazol-1- yl)pyridin-3-yl]-1H-indole | 279 |
| 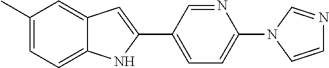 | 2-[6-(1H-imidazol-1- yl)pyridin-3-yl]-5-methyl-1H- indole | 275 |
| 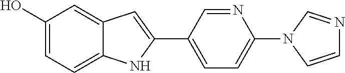 | 2-[6-(1H-imidazol-1- yl)pyridin-3-yl]-1H-indol-5-ol | 277 |
| 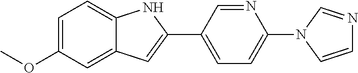 | 2-[6-(1H-imidazol-1- yl)pyridin-3-yl]-5-methoxy- 1H-indole | 291 |
| 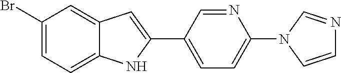 | 5-bromo-2-[6-(1H-imidazol- 1-yl)pyridin-3-yl]-1H-indole | 339 |
| 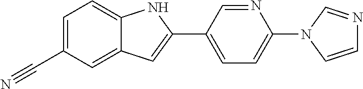 | 2-[6-(1H-imidazol-1- yl)pyridin-3-yl]-1H-indole-5- carbonitrile | 286 |
| 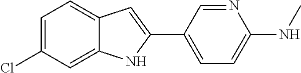 | 5-(6-chloro-1H-indol-2-yl)-N- methylpyridin-2-amine | 258 |
| 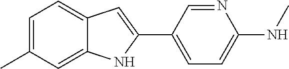 | N-methyl-5-(6-methyl-1H- indol-2-yl)pyridin-2-amine | 238 |
| 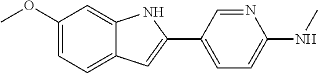 | 5-(6-methoxy-1H-indol-2-yl)- N-methylpyridin-2-amine | 254 |
| 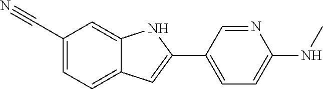 | 2-[6-(methylamino)pyridin-3- yl]-1H-indole-6-carbonitrile | 249 |
| 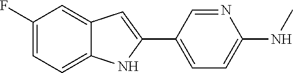 | 5-(5-fluoro-1H-indol-2-yl)-N- methylpyridin-2-amine | 242 |
| 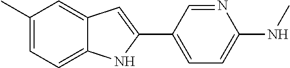 | N-methyl-5-(5-methyl-1H- indol-2-yl)pyridin-2-amine | 238 |
| 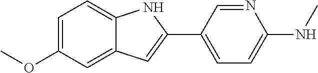 | 5-(5-methoxy-1H-indol-2-yl)- N-methylpyridin-2-amine | 254 |
| 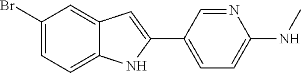 | 5-(5-bromo-1H-indol-2-yl)- N-methylpyridin-2-amine | 302 |
| 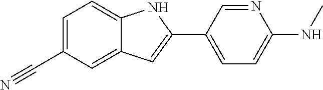 | 2-[6-(methylamino)pyridin-3- yl]-1H-indole-5-carbonitrile | 249 |
| 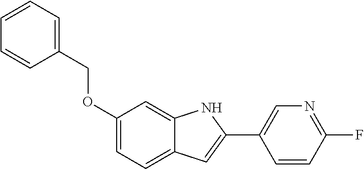 | 6-(benzyloxy)-2-(6- fluoropyridin-3-yl)-1H-indole | 319 |
| 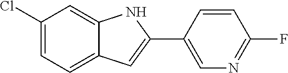 | 6-chloro-2-(6-fluoropyridin- 3-yl)-1H-indole | 247 |
| 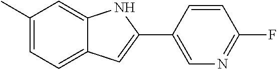 | 2-(6-fluoropyridin-3-yl)-6- methyl-1H-indole | 227 |
| 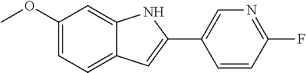 | 2-(6-fluoropyridin-3-yl)-6- methoxy-1H-indole | 243 |
| 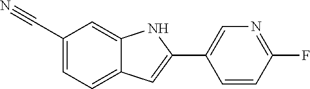 | 2-(6-fluoropyridin-3-yl)-1H- indole-6-carbonitrile | 238 |
| 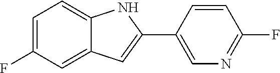 | 5-fluoro-2-(6-fluoropyridin-3- yl)-1H-indole | 231 |
| 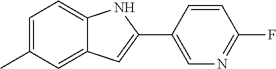 | 2-(6-fluoropyridin-3-yl)-5- methyl-1H-indole | 227 |
| 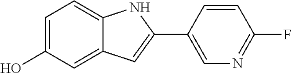 | 2-(6-fluoropyridin-3-yl)-1H- indol-5-ol | 229 |
| 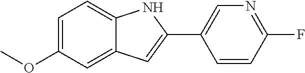 | 2-(6-fluoropyridin-3-yl)-5- methoxy-1H-indole | 243 |
| 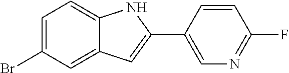 | 5-bromo-2-(6-fluoropyridin- 3-yl)-1H-indole | 291 |
| 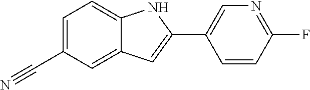 | 2-(6-fluoropyridin-3-yl)-1H- indole-5-carbonitrile | 238 |
| 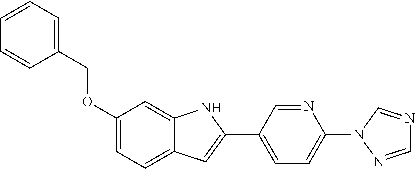 | 6-(benzyloxy)-2-[6-(1H- 1,2,4-triazol-1-yl)pyridin-3-yl]- 1H-indole | 368 |
| 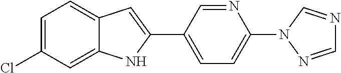 | 6-chloro-2-[6-(1H-1,2,4- triazol-1-yl)pyridin-3-yl]-1H- indole | 296 |
| 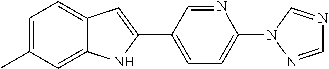 | 6-methyl-2-[6-(1H-1,2,4- triazol-1-yl)pyridin-3-yl]-1H- indole | 276 |
| 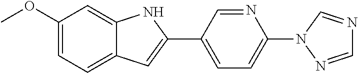 | 6-methoxy-2-[6-(1H-1,2,4- triazol-1-yl)pyridin-3-yl]-1H- indole | 292 |
| 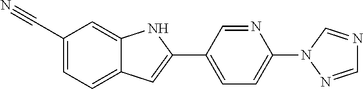 | 2-[6-(1H-1,2,4-triazol-1- yl)pyridin-3-yl]-1H-indole-6- carbonitrile | 287 |
| 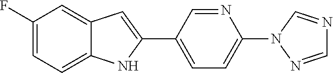 | 5-fluoro-2-[6-(1H-1,2,4- triazol-1-yl)pyridin-3-yl]-1H- indole | 280 |
| 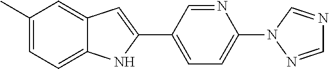 | 5-methyl-2-[6-(1H-1,2,4- triazol-1-yl)pyridin-3-yl]-1H- indole | 276 |
| 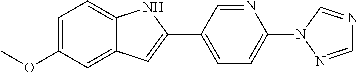 | 5-methoxy-2-[6-(1H-1,2,4- triazol-1-yl)pyridin-3-yl]-1H- indole | 292 |
| 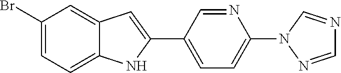 | 5-bromo-2-[6-(1H-1,2,4- triazol-1-yl)pyridin-3-yl]-1H- indole | 340 |
| 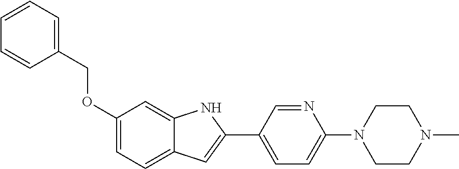 | 6-(benzyloxy)-2-[6-(4- methylpiperazin-1-yl)pyridin- 3-yl]-1H-indole | 399 |
| 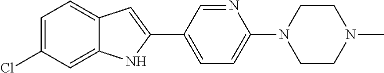 | 6-chloro-2-[6-(4- methylpiperazin-1-yl)pyridin- 3-yl]-1H-indole | 327 |
| 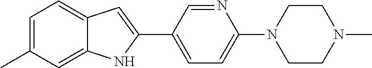 | 6-methyl-2-[6-(4- methylpiperazin-1-yl)pyridin- 3-yl]-1H-indole | 307 |
| 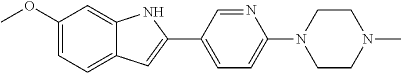 | 6-methoxy-2-[6-(4- methylpiperazin-1-yl)pyridin- 3-yl]-1H-indole | 323 |
| 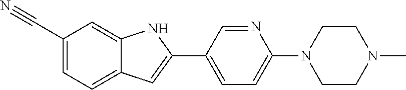 | 2-[6-(4-methylpiperazin-1- yl)pyridin-3-yl]-1H-indole-6- carbonitrile | 318 |
| 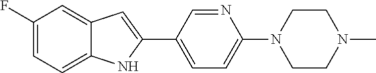 | 5-fluoro-2-[6-(4- methylpiperazin-1-yl)pyridin- 3-yl]-1H-indole | 311 |
| 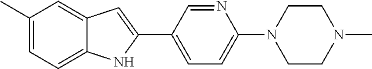 | 5-methyl-2-[6-(4- methylpiperazin-1-yl)pyridin- 3-yl]-1H-indole | 307 |
| 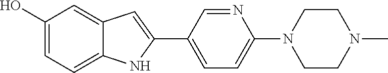 | 2-[6-(4-methylpiperazin-1- yl)pyridin-3-yl]-1H-indol-5-ol | 309 |
| 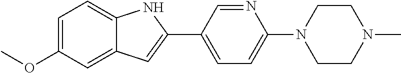 | 5-methoxy-2-[6-(4- methylpiperazin-1-yl)pyridin- 3-yl]-1H-indole | 323 |
| 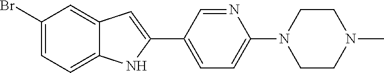 | 5-bromo-2-[6-(4- methylpiperazin-1-yl)pyridin- 3-yl]-1H-indole | 371 |
| 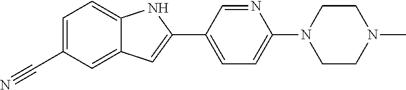 | 2-[6-(4-methylpiperazin-1- yl)pyridin-3-yl)-1H-indole-5- carbonitrile | 318 |
| 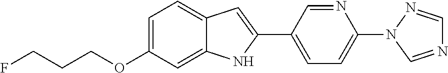 | 6-(3-fiuoropropoxy)-2-[6- (1H-1,2,4-triazol-1-yl)pyridin- 3-yl]-1H-indole | 338 |
| 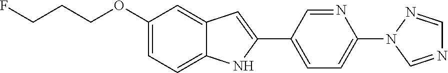 | 5-(3-fluoropropoxy)-2-[6- (1H-1,2,4-triazol-1-yl)pyridin- 3-yl]-1H-indole | 338 |
| 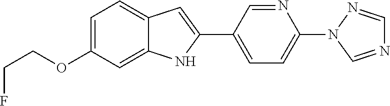 | 6-(2-fluoroethoxy)-2-[6-(1H- 1,2,4-triazol-1-yl)pyridin-3-yl]- 1H-indole | 324 |
| 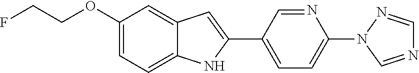 | 5-(2-fluoroethoxy)-2-[6-(1H- 1,2,4-triazol-1H-yl)pyridin-3-yl]- 1H-indole | 324 |
| 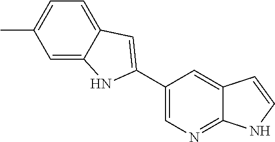 | 5-(6-methyl-1H-indol-2-yl)- 1H-pyrrolo[2,3-b]pyridine | 248 |
| 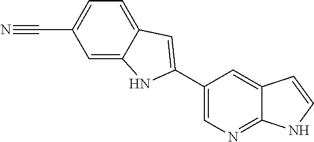 | 2-(1H-pyrrolo[2,3-b]pyridin- 5-yl)-1H-indole-6-carbonitrile | 259 |
| 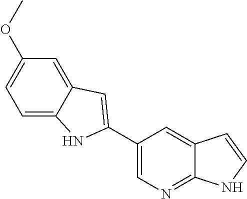 | 5-(5-methoxy-1H-indol-2-yl)- 1H-pyrrolo[2,3-b]pyridine | 264 |
| 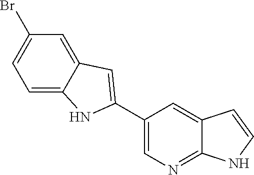 | 5-(5-bromo-1H-indol-2-yl)- 1H-pyrrolo[2,3-b]pyridine | 312 |
| 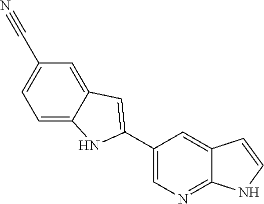 | 2-(1H-pyrrolo[2,3-b]pyridin- 5-yl)-1H-indole-5-carbonitrile | 259 |
| 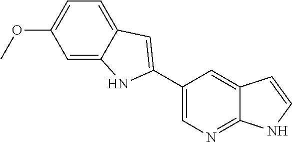 | 5-(6-methoxy-1H-indol-2-yl)- 1H-pyrrolo[2,3-b]pyridine | 264 |
| 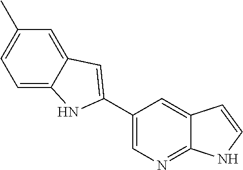 | 5-(5-methyl-1H-indol-2-yl)- 1H-pyrrolo[2,3-b]pyridine | 248 |
| 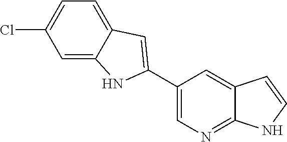 | 5-(6-chloro-1H-indol-2-yl)- 1H-pyrrolo[2,3-b]pyridine | 268 |

or a pharmaceutically acceptable salt, solvate or in vivo hydrolysable ester thereof.

The present invention also relates to methods for measuring effects of the compounds, by measuring changes of amyloid plaque level in living patients. More specifically, the present invention relates to a method of using the compounds of this invention as tracers in positron emission tomography (PET) imaging to study amyloid deposits in brain in vivo to allow diagnosis of Alzheimer's disease. Thus, the present invention relates to use of the novel amyloid binding compounds as a diagnostic. The invention further relates to the use of the novel amyloid binding compounds in the manufacture of a medicament for treating Alzeheimer's disease. The invention further relates to a method of measuring clinical efficacy of Alzheimer's disease therapeutic agents. Specifically, the present invention relates to novel aryl or heteroaryl substituted indole derivatives, compositions, and therapeutic uses and processes for making such compounds. The invention is further directed to ²H, ³H, ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁶Br, ⁷⁷Br, ¹²³I, ¹²⁴I and ¹³¹I, preferably ¹¹C, ¹³C, ¹⁴C, ¹⁸F, ¹⁵O, ¹³N, ³⁵S, ²H, and ³H, more preferably ¹¹C, and ¹⁸F isotopically labeled aryl or heteroaryl substituted indole derivative compounds, compositions and methods of their preparation. The present invention also relates to non-toxic amyloid binding compounds that can rapidly cross the blood brain barrier, have low non-specific binding properties and rapidly clear from the system.

The compounds of the present invention may have asymmetric centers, chiral axes and chiral planes, and occur as racemates, racemic mixtures, and as individual diastereomers, with all possible isomers, including optical isomers, being included in the present invention. (See E. L. Eliel and S. H. Wilen Stereochemistry of Carbon Compounds (John Wiley and Sons, New York 1994), in particular pages 1119-1190)

When any variable (e.g. aryl, heterocycle, R^{1a}, R⁶ etc.) occurs more than one time in any constituent, its definition on each occurrence is independent at every other occurrence. Also, combinations of substituents/or variables are permissible only if such combinations result in stable compounds.

In addition, the compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof.

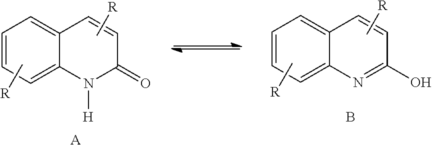

As used herein, “alkyl” is intended to include both branched and straight-chain saturated aliphatic hydrocarbon groups having the specified number of carbon atoms; “alkoxy” represents an alkyl group of indicated number of carbon atoms attached through an oxygen bridge. “Halogen” or “halo” as used herein means fluoro, chloro, bromo and iodo.

Preferably, alkenyl is C₂-C₆ alkenyl.

Preferably, alkynyl is C₂-C₆ alkynyl.

As used herein, “cycloalkyl” is intended to include cyclic saturated aliphatic hydrocarbon groups having the specified number of carbon atoms. Preferably, cycloalkyl is C₃-C₁₀ cycloalkyl. Examples of such cycloalkyl elements include, but are not limited to, cyclopropyl, cyclobutyl, cyclopentyl, cyclohexyl and cycloheptyl.

As used herein, “aryl” is intended to mean any stable monocyelic or bicyclic carbon ring of up to 7 members in each ring, wherein at least one ring is aromatic. Examples of such aryl elements include phenyl, naphthyl, tetrahydronaphthyl, indanyl, biphenyl, phenanthryl, anthryl or acenaphthyl.

The term heterocyclyl, heterocycle or heterocyclic, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. The term heterocyclyl, heterocycle or heterocyclic includes heteroaryl moieties. Examples of such heterocyclic elements include, but are not limited to, azepinyl, benzodioxolyl, benzimidazolyl, benzisoxazolyl, benzofurazanyl, benzopyranyl, benzothiopyranyl, benzofuryl, benzothiazolyl, benzothienyl, benzotriazolyly, benzoxazolyl, chromanyl, cinnolinyl, dihydrobenzofuryl, dihydrobenzothienyl, dihydrobenzothiopyranyl, dihydrobenzothiopyranyl sulfone, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyrazolopyridinyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, thienyl, and triazolyl. An embodiment of the examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, 2-pyridinonyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, thienyl and triazolyl.

Preferably, heterocycle is selected from 2-azepinonyl, benzimidazolyl, 2-diazapinonyl, imidazolyl, 2-imidazolidinonyl, indolyl, isoquinolinyl, morpholinyl, piperidyl, piperazinyl, pyridyl, pyrrolidinyl, 2-piperidinonyl, 2-pyrimidinonyl, 2-pyrollidinonyl, quinolinyl, tetrahydrofuryl, tetrahydroisoquinolinyl, thienyl and triazolyl.

As used herein, “heteroaryl” is intended to mean any stable monocyclic or bicyclic carbon ring of up to 7 members in each ring, wherein at least one ring is aromatic and wherein from one to four carbon atoms are replaced by heteroatoms selected from the group consisting of N, O, and S. Examples of such heterocyclic elements include, but are not limited to, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolyl, indolinyl, indolyl, isochromanyl, isoindolinyl, isoquinolinyl, isothiazolyl, naphthyridinyl, oxadiazolyl, pyridyl, pyrazinyl, pyrazolyl, pyridazinyl, pyrimidinyl, pyrrolyl, quinazolinyl, quinolinyl, quinoxalinyl, tetrahydroisoquinolinyl, tetrahydroquinolinyl, thiazolyl, thienofuryl, thienothienyl, thienyl and triazolyl.

As used herein, unless otherwise specifically defined, substituted alkyl, substituted cycloalkyl, substituted aroyl, substituted aryl, substituted heteroaroyl, substituted heteroaryl, substituted arylsulfonyl, substituted heteroaryl-sulfonyl and substituted heterocycle include moieties containing from 1 to 3 substituents in addition to the point of attachment to the rest of the compound. Preferably, such substituents are selected from the group which includes but is not limited to F, Cl, Br, CF₃, NH₂, N(C₁-C₆ alkyl)₂, NO₂, CN, (C₁-C₆ alkyl)O—, (aryl)O—, —OH, (C₁-C₆ alkyl)S(O)ₘ—, (C₁-C₆ alkyl)C(O)NH—, H₂N—C(NH)—, (C₁-C₆ alkyl)C(O)—, (C₁-C₆ alkyl)OC(O)—, (C₁-C₆ alkyl)OC(O)NH—, phenyl, pyridyl, imidazolyl, oxazolyl, isoxazolyl, thiazolyl, thienyl, furyl, isothiazolyl and C₁-C₂₀ alkyl.

As used herein, “in vivo hydrolysable precursors” means an in vivo hydrolysable (or cleavable) ester of a compound of formula I that contains a carboxy or a hydroxy group. For example amino acid esters, C1-6 alkoxymethyl esters like methoxymethyl; C₁₋₆ alkanoyloxymethyl esters like pivaloyloxymethyl; C₃₋₈cycloalkoxycarbonyloxy, C1-6alkyl esters like 1-cyclohexylearbonyloxyethyl, acetoxymethoxy, or phosphoramidic cyclic esters.

Examples of an “effective amount” include amounts that enable imaging of amyloid deposit(s) in vivo, that yield acceptable toxicity and bioavailability levels for pharmaceutical use, and/or prevent cell degeneration and toxicity associated with fibril formation.

For use in medicine, the salts of the compounds of formula I will be pharmaceutically acceptable salts. Other salts may, however, be useful in the preparation of the compounds according to the invention or of their pharmaceutically acceptable salts. When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The preparation of the pharmaceutically acceptable salts described above and other typical pharmaceutically acceptable salts is more fully described by Berg et al., “Pharmaceutical Salts,” J. Pharm. Sci., 1977:66:1-19.

As indicated herein the present invention includes isotopically labeled compounds of the invention. An “isotopically-labeled”, “radio-labeled”, “tracer”, “labeled tracer” “radioligand” or “detectable amyloid binding” compound, is a compound where one or more atoms are replaced or substituted by an atom having an atomic mass or mass number different from the atomic mass or mass number typically found in nature (i.e., naturally occurring). Suitable radionuclides (i.e. “detectable isotopes”) that may be incorporated in compounds of the present invention include but are not limited to ²H, ³H, ¹¹C, ¹³C, ¹⁴C, ¹³N, ¹⁵N, ¹⁵O, ¹⁷O, ¹⁸O, ¹⁸_{F,} ³⁵S, ³⁶Cl, ⁸²Br, ⁷⁶Br, ⁷⁷Br, ¹²³I, ¹²⁴I and ¹³¹I. The isotopically labeled compounds of the invention need only to be enriched with a detectable isotope to, or above, the degree which allows detection with a technique suitable for the particular application. The radionuclide that is incorporated in the instant radiolabeled compounds will depend on the specific application of that radiolabeled compound. In another embodiment of the invention the radionuclides are represented by ¹¹C, ¹³C, ¹⁴C, ¹⁸F, ¹⁵O, ¹³N, ³⁵S, ²H, and ³H, preferably ¹¹C, and ¹⁸F.

This invention further relates to a pharmaceutical composition comprising an effective amount of at least one compound of formula I and a pharmaceutically acceptable carrier. The composition may comprise, but is not limited to, one or more buffering agents, wetting agents, emulsifiers, suspending agents, lubricants, adsorbents, surfactants, preservatives and the like. The composition may be formulated as a solid, liquid, gel or suspension for oral administration (e.g., drench, bolus, tablet, powder, capsule, mouth spray, emulsion); parenteral administration (e.g., subcutaneous, intramuscular, intravenous, epidural injection); topical application (e.g., cream, ointment, controlled-released patch, spray); intravaginal, intrarectal, transdermal, ocular, or nasal administration.

This invention provides radiolabeled aryl or heteroaryl substituted indole derivatives as amyloid imaging agents and synthetic precursor compounds from which they are prepared. The compounds formula I are active against age-related diseases such as

Alzheimer, as well as other pathologies such as Downs syndrome and beta-amyloid angiopathy. The compounds of this invention may also be used in combination with a broad range of cognition deficit enhancement agents. Thus, in another embodiment of this invention a compound of formula (I) or a pharmaceutically acceptable salt, solvate or in vivo hydrolysable ester thereof, or a pharmaceutical composition or formulation comprising a compound of formula (I) is administered concurrently, simultaneously, sequentially or separately with another pharmaceutically active compound or compounds used in Alzheimer's therapies including for example donepezil, memantine, tacrine and equivalents and pharmaceutically active isomer(s) and metabolite(s) thereof.

This invention further relates to a method of treating or preventing an Aβ-related pathology in a patient comprising administering a therapeutically effective amount of a compound of formula I. This invention also provides a method for treating neurodegenerative disorders such as dementia, Cognitive Deficit in Schizophrenia, Mild Cognitive Impairment, Age Associated Memory Impairment, Age-Related Cognitive Decline, and the like.

An ultimate objective of the present invention is to provide a radiopharmaceutical agent, useful in PET imaging that has high specific radioactivity and high target tissue selectivity by virtue of its high affinity for amyloid plaques. The tissue selectivity is capable of further enhancement by coupling this highly selective radiopharmaceutical with targeting agents, such as microparticles.

In accordance with the present invention, the most preferred method for imaging beta-amyloid plaque in a patient, wherein an isotopically labeled novel aryl or heteroaryl substituted indole derivative is employed as the imaging agent, comprises the following steps: the patient is placed in a supine position in the PET camera, a sufficient amount (about 10 mCi) of an isotopically labeled aryl or heteroaryl substituted indole derivative is administered to the brain tissue of the patient. An emission scan of the cerebral region is performed. The technique for performing an emission scan of the head is well known to those of skill in the art. PET techniques are described in Freeman et al., Freeman and Johnson's Clinical Radionuclide Imaging. 3rd. Ed. Vol. 1 (1984); Grune & Stratton, New York; Ennis et Q. Vascular Radionuclide Imaging: A Clinical Atlas, John Wiley & Sons, New York (1983).

The term “labeled tracer” refers to any molecule which can be used to follow or detect a defined activity in vivo, for example, a preferred tracer is one that accumulates in the regions where beta-amyloid plaque may be found. Preferably, the labeled tracer is one that can be viewed in a living experimental animal, healthy human or patient (referred to as a subject), for example, by positron emission tomograph (PET) scanning. Suitable labels include, but are not limited to radioisotopes, fluorochromes, chemiluminescent compounds, dyes, and proteins, including enzymes.

The present invention also provides methods of determining in vivo activity of an enzyme or other molecule. More specifically, a tracer, which specifically tracks the targeted activity, is selected and labeled. In a preferred embodiment, the tracer tracks binding activity of amyloid Aβ-peptide in the brain and central nervous system. The tracer provides the means to evaluate various neuronal processes, including fast excitatory synaptic transmission, regulation of neurotransmitter release, and long-term potentiation. The present invention gives researchers the means to study the biochemical mechanisms of pain, anxiety/depression, drug addiction and withdrawal, disorders of the basal ganglia, eating disorders, obesity, long-term depression, learning and memory, developmental synaptic plasticity, hypoxic-ischemic damage and neuronal cell death, epileptic seizures, visual processing, as well as the pathogenesis of several neurodegenerative disorders.

Biomarkers of Alzheimer's disease state, prognosis and progression will all be useful for general diagnostic utilities as well as for clinical development plans for therapeutic agents for Alzheimer's disease. The present invention will provide biomarker information as patients are enrolled in clinical trials for new Alzheimer's treatments to assist in patient selection and assignment to cohorts. The present invention will serve as one of the biomarkers of disease state in order to get the correct patients into the proper PhIIb trial cohort. In addition, the present invention can serve as one marker of disease prognosis as an entry inclusion criterion in order to enhance the probability that the disease will progress in the placebo treatment arm, an issue that has plagued recent AD clinical trials. Finally, the present invention can serve as one biomarker of disease progression to monitor the clinical course of patients on therapy and could provide an independent biomarker measure of treatment response by a therapeutic drug.

Means of detecting labels are well known to those skilled in the art. For example, isotopic labels may be detected using imaging techniques, photographic film or scintillation counters. In a preferred embodiment, the label is detected in vivo in the brain of the subject by imaging techniques, for example positron emission tomography (PET).

The labeled compound of the invention preferably contains at least one radionuclide as a label. Positron-emitting radionuclides are all candidates for usage. In the context of this invention the radionuclide is preferably selected from ¹¹C, ¹³C, ¹⁴C, ¹⁸F, ¹⁵O, ¹³N, ³⁵S, ²H, and ³H, more preferably from ¹¹C, and ¹⁸F.

The tracer can be selected in accordance with the detection method chosen. Before conducting the method of the present invention, a diagnostically effective amount of a labeled or unlabeled compound of the invention is administered to a living body, including a human.

The diagnostically effective amount of the labeled or unlabeled compound of the invention to be administered before conducting the in-vivo method for the present invention is within a range of from 0.1 ng to 100 mg per kg body weight, preferably within a range of from 1 ng to 10 mg per kg body weight.

In accordance with another embodiment of the present invention, there are provided methods for the preparation of heterocyclic compounds as described above. For example, the heterocyclic compounds described above can be prepared using synthetic chemistry techniques well known in the art (see Comprehensive Heterocyclic Chemistry, Katritzky, A. R. and Rees, C. W. eds., Pergamon Press, Oxford, 1984) from a precursor of the substituted heterocycle of Formula 1 as outlined below. The isotopically labeled compounds of this invention are prepared by incorporating an isotope such as ¹¹C, ¹³C, ¹⁴C, ¹⁸F, ¹⁵O, ¹³N, ³⁵S, ²H, and ³H into the substrate molecule. This is accomplished by utilizing reagents that have had one or more of the atoms contained therein made radioactive by placing them in a source of radioactivity such as a nuclear reactor, a cyclotron and the like. Additionally many isotopically labeled reagents, such as ²H₂O, ³H₃Cl, ¹⁴C₆H₅Br, ClCH₂¹⁴COCl and the like, are commercially available. The isotopically labeled reagents are then used in standard organic chemistry synthetic techniques to incorporate the isotope atom, or atoms, into a compound of Formula I as described below. The following Schemes illustrate how to make the compounds of formula I.

Abbreviations used in the description of the chemistry and in the Examples that follow are:

CH₂Cl₂ dichloromethane

dppf 1,1′-bis(diphenylphosphino)ferrocene

Boc tert-butoxycarbonyl

DIEA diisopropylethylamine

PMB 4-methoxy-benzyl

PMBBr 4-methoxy-benzyl bromide

THF tetrahydrofuran

TFA trifluoroacteic acid

MeOH methanol

PS-PPh3 polystyrene triphenyphosphine

DMF N,N-dimethylformamide

DMA N,N-dimethylacetamide

EtOAc ethyl acetate

AD Alzheimer's Disease

NMR Nuclear Magnetic Resonance

DMSO dimethyl sulfoxide

Several methods for preparing the compounds of this invention are illustrated in the following Schemes and Examples. Starting materials and the requisite intermediates are in some cases commercially available, or can be prepared according to literature procedures or as illustrated herein. Pd ENCat™ 30 was purchased from Sigma-Aldrich, P.O. Box 2060, Milwaukee, Wis. 53201.

The compounds of this invention may be prepared by employing reactions as shown in the following schemes, in addition to other standard manipulations that are known in the literature or exemplified in the experimental procedures. Substituent numbering as shown in the schemes does not necessarily correlate to that used in the claims and often, for clarity, a single substituent is shown attached to the compound where multiple substituents are allowed under the definitions hereinabove. Reactions used to generate the compounds of this invention are prepared by employing reactions as shown in the schemes and examples herein, in addition to other standard manipulations such as ester hydrolysis, cleavage of protecting groups, etc., as may be known in the literature or exemplified in the experimental procedures.

In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

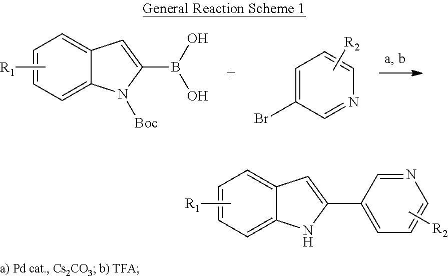

As illustrated in General Reaction Scheme 1, a suitably substituted 2-indoleboronic acid is reacted with substituted heterocyclic halide under palladium catalyzed coupling conditions to provide the corresponding Suzuki coupling product, which is deprotected under the action of TFA. In this instance, all starting materials are commercially available.

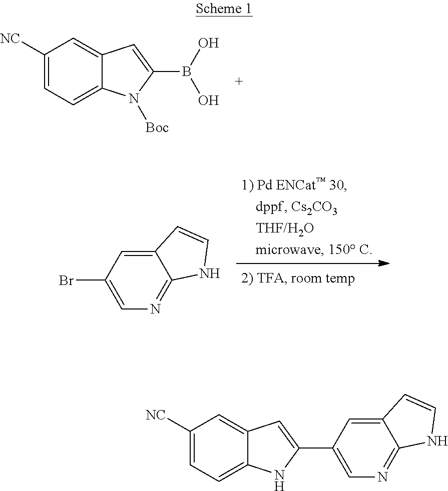

### EXAMPLE 1

### 2-(1H-pyrrolo[2,3-b]pyridin-5-yl)-1H-indole-5-carbonitrile

A mixture containing 5-cyano-indole-1-Boc-2-boronic acid (45 mg, 0.25 mmol), 5-bramo-1H-pyrrolo[2,3-b]pyridine (50 mg, 0.25 mmol), Pd ENCat™ 30 (32 mg, 0.013 mmol), dppf (7 mg, 0.013 mmol), 1 M aqueous Cs₂CO₃ (0.85 mL, 0.85 mmol), and THF (1.0 mL) was microwaved to 150° C. for 10 minutes in a sealed microwave tube. After cooling to room temperature, the aqueous layer was removed by pipette and the remaining organics were filtered and concentrated. The remaining residue was treated with TFA (1 mL) for 2 h then concentrated, affording a residue which was purified by reversed phase HPLC to afford 2-(1H-pyrrolo[2,3-b]pyridin-5-yl)-1H-indole-5-carbonitrile (4.4 mg, 0.017 mmol, 6.8% yield). ES MS (M+H⁺)=259; ¹H NMR (499 MHz, DMSO): δ 12.16 (1H, s), 11.82 (1H, s), 8.79 (1H, d, J=2.14 Hz), 8.44 (1H, d, J=2.10 Hz), 8.05 (1H, s), 7.56-7.53 (2H, m), 7.43 (1H, dd, J=8.37, 1.57 Hz), 7.05 (1H, s), 6.55 (1H, dd, J=3.42, 1.65 Hz); HRMS m/z 259.0989 (C₁₆H₁₀N₄+H⁺ requires 259.0978).

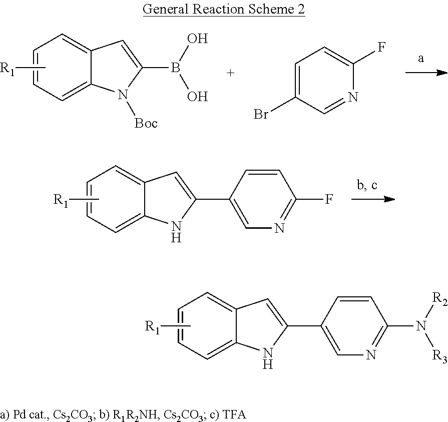

As illustrated in General Reaction Scheme 1, a suitably substituted 2-indoleboronic acid is reacted with 5-bromo-2-fluoropyridine under palladium catalyzed coupling conditions to provide the corresponding Suzuki coupling product, which is deprotected under the action of TFA. The resulting product is then reacted with amines or nitrogen containing heterocycles under basic conditions to afford the final product.

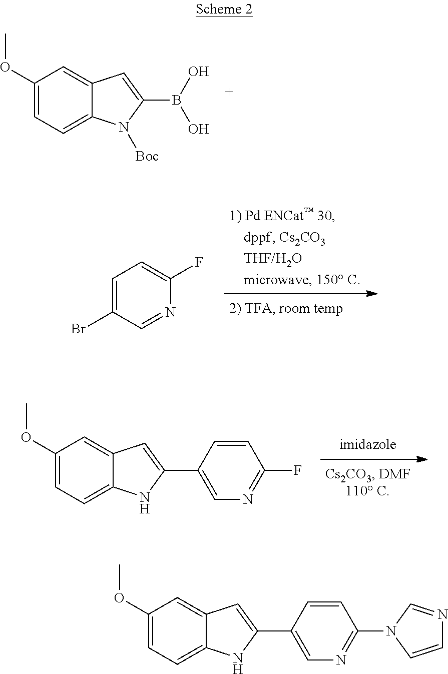

### EXAMPLE 2

### 2-[6-(1H-imidazol-1-yl)pyridin-3-yl]-5-methoxy-1H-indole

### Step 1: 2-(6-Fluoro-pyridin-3-yl)-5-methoxy-1H-indole

A mixture containing 5-methoxy-indole-1-Boc-2-boronic acid (235 mg, 0.85 mmol), 5-bromo-2-fluoropyridine (150 mg, 0.85 mmol), Pd ENCat™ 30 (107 mg, 0.043 mmol), dppf (24 mg, 0.043 mmol), 1 M aqueous Cs₂CO₃ (0.85 mL, 0.85 mmol), and dioxane (2.1 mL) was microwaved to 150° C. for 15 minutes in a sealed microwave tube. After cooling to room temperature, the aqueous layer was removed by pipette and the remaining organics were filtered. The remaining solution was divided evenly into 5 vials and concentrated, leaving crude residues, which were each treated with TFA (1 mL) for 2 h. Concentration afforded 5 equal batches of a crude residue containing 2-(6-Fluoro-pyridin-3-yl)-5-methoxy-1H-indole which was used in subsequent steps without further purification. ES MS (M+H⁺)=243.

### Step 2: 2-[6-(1H-imidazol-1-yl)pyridin-3-yl]-5-methoxy-1H-indole

⅕^{th} of the crude reaction product from Step 1 was dissolved in 1 mL of DMF, and the resulting solution was treated with imidazole (50 mg, 0.73 mmol) and Cs₂CO₃ (50 mg, 0.15 mmol), then heated to 110° C. in a sealed vial for 16 h. After cooling to room temperature, the reaction mixture was filtered then concentrated and the crude residue was purified by reversed phase HPLC to afford 2-[6-(1H-imidazol-1-yl)pyridin-3-yl]-5-methoxy-1H-indole (14 mg, 0.048 mmol, 28% yield). ES MS (M+H⁺)=291; ¹H NMR (499 MHz, DMSO): δ 11.62 (1H, s), 9.38 (1H, s), 9.05 (1H, d, J=2.35 Hz), 8.50 (1H, dd, J=8.58, 2.40 Hz), 8.31 (1H, s), 8.05 (1H, d, J=8.59 Hz), 7.62 (1H, s), 7.34 (1H, d, J=8.78 Hz), 7.08-7.05 (2H, m), 6.82 (1H, dd, J=8.77, 2.44 Hz), 3.78 (3H, s); HRMS m/z 291.1237 (C₁₇H₁₄N₄O+H⁺ requires 291.1240).

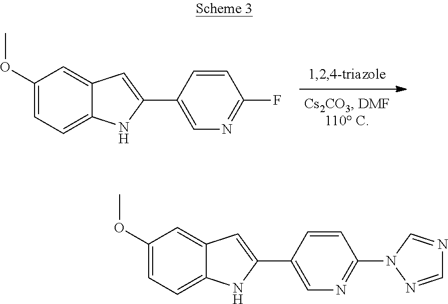

### EXAMPLE 3

### 6-Methoxy-2-[6-(1H-1,2,4-triazol-1-yl)pyridin-3-yl]-1H-indole

⅕^{th} of the crude reaction product containing 2-(6-fluoro-pyridin-3-yl)-5-methoxy-1H-indole from the first step of Example 2 was dissolved in 1 mL of DMF, and the resulting solution was treated with 1,2,4-triazole (50 mg, 0.72 mmol) and Cs₂CO₃ (50 mg, 0.15 mmol), then heated to 110° C. in a sealed vial for 16 h. After cooling to room temperature, the reaction mixture was filtered then concentrated and the crude residue was purified by reversed phase HPLC to afford 6-methoxy-2-[6-(1H-1,2,4-triazol-1-yl)pyridin-3-yl]-1H-indole (7.5 mg, 0.025 mmol, 15% yield). ES MS (M+H⁺)=292; ¹H NMR (499 MHz, DMSO): δ 11.62 (1H, s), 9.41 (1H, s), 9.02 (1H, d, J=2.34 Hz), 8.46 (1H, dd, J=8.55, 2.39 Hz), 8.34 (1H, s), 7.96 (1H, d, J=8.54 Hz), 7.34 (1H, d, J=8.77 Hz), 7.07 (1H, d, J=2.42 Hz), 7.03 (1H, d, J=1.90 Hz), 6.81 (1H, dd, J=8.76, 2.45 Hz), 3.78 (3H, s); HRMS m/z 292.1195 (C₁₆H₁₃N₅O+H⁺ requires 292.1193).

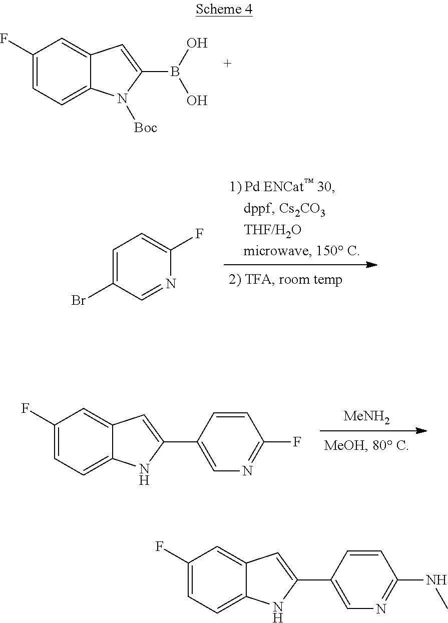

EXAMPLE 4

### 5-(5-Fluoro-1H-indol-2-yl)-N-methylpyridin-2-amine

### Step 1: 5-Fluoro-2-(6-fluoro-pyridin-3-yl)-1H-indole

A mixture containing 5-fluoro-indole-1-Boc-2-boronic acid (237 mg, 0.85 mmol), 5-bromo-2-fluoropyridine (150 mg, 0.85 mmol), Pd ENCat™ 30 (107 mg, 0.043 mmol), dppf (24 mg, 0.043 mmol), 1 M aqueous Cs₂CO₃ (0.85 mL, 0.85 mmol), and dioxane (2.1 mL) was microwaved to 150° C. for 15 minutes in a sealed microwave tube. After cooling to room temperature, the aqueous layer was removed by pipette and the remaining organics were filtered. The remaining solution was divided evenly into 5 vials and concentrated, leaving crude residues, which were each treated with TFA (1 mL) for 2 h. Concentration afforded 5 equal batches of a crude residue containing 2-(6-fluoro-pyridin-3-yl)-5-fluoro-1H-indole which was used in subsequent steps without further purification. ES MS (M+H⁺)=231.

### Step 2: 5-(5-Fluoro-1H-indol-2-yl)-N-methylpyridin-2-amine

⅕^{th} of the crude reaction product containing 2-(6-fluoro-pyridin-3-yl)-5-fluoro-1H-indole from Step 1 was treated with 33% methylamine in MeOH (2 mL) and the resulting solution was heated to 80° C. in a sealed vial for 16 h. After cooling to room temperature, the reaction mixture was filtered then concentrated and the crude residue was purified by reversed phase HPLC to afford 5-(5-Fluoro-1H-indol-2-yl)-N-methylpyridin-2-amine (6.7 mg, 0.028 mmol, 16% yield). ES MS (M+H⁺)=242; ¹H NMR (499 MHz, DMSO): δ 11.63 (1H, s), 8.37 (1H, s), 8.17 (1H, s), 7.37 (1H, dd, J=8.81, 4.54 Hz), 7.27 (1H, dd, J=9.86, 2.53 Hz), 6.97-6.91 (2H, m), 6.85 (1H, s), 2.93 (3H, s); HRMS m/z 242.1085 (C₁₄H₁₂FN₃+H⁺ requires 242.1088).

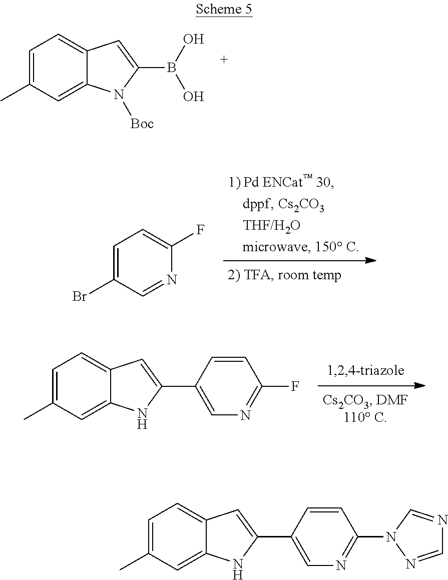

### EXAMPLE 5

### 6-methyl-2-[6-(1H-1,2,4-triazol-1-yl)pyridin-3-yl]-1H-indole

### Step 1: 2-(6-Fluoro-pyridin-3-yl)-6-methyl-1H-indole

A mixture containing 6-methyl-indole-1-Boc-2-boronic acid (234 mg, 0.85 mmol), 5-bromo-2-fluoropyridine (150 mg, 0.85 mmol), Pd ENCat™ 30 (107 mg, 0.043 mmol), dppf (24 mg, 0.043 mmol), 1 M aqueous Cs₂CO₃ (0.85 mL, 0.85 mmol), and dioxane (2.1 mL) was microwaved to 150° C. for 15 minutes in a sealed microwave tube. After cooling to room temperature, the aqueous layer was removed by pipette and the remaining organics were filtered. The remaining solution was divided evenly into 5 vials and concentrated, leaving crude residues, which were each treated with TFA (1 mL) for 2 h. Concentration afforded 5 equal batches of a crude residue containing 2-(6-fluoro-pyridin-3-yl)-6-methyl-1H-indole which was used in subsequent steps without further purification. ES MS (M+H⁺)=227.

### Step 2: 6-methyl-2-[6-(1H-1,2,4-triazol-1-yl)pyridin-3-yl]-1H-indole

⅕^{th} of the crude reaction product containing 2-(6-fluoro-pyridin-3-yl)-6-methyl-1H-indole from the first step of Example 4 was dissolved in 1 mL of DMF, and the resulting solution was treated with 1,2,4-triazole (50 mg, 0.72 mmol) and Cs₂CO₃ (50 mg, 0.15 mmol), then heated to 110° C. in a sealed vial for 16 h. After cooling to room temperature, the reaction mixture was filtered then concentrated and the crude residue was purified by reversed phase HPLC to afford 6-methyl-2-[6-(1H-1,2,4-triazol-1-yl)pyridin-3-yl]-1H-indole (7.5 mg, 0.025 mmol, 15% yield). ES MS (M+H⁺)=276; ¹H NMR (499 MHz, DMSO): δ 11.59 (1H, s), 9.41 (1H, s), 9.02 (1H, d, J=2.33 Hz), 8.47 (1H, dd, J=8.55, 2.39 Hz), 8.33 (1H, s), 7.95 (1H, d, J=8.54 Hz), 7.46 (1H, d, J=8.05 Hz), 7.23 (1H, s), 7.06 (1H, d, J=1.90 Hz), 6.88 (1H, d, J=8.10 Hz), 2.42 (3H, s); HRMS m/z 276.1246 (C₁₆H₁₃N₅+H⁺ requires 276.1244).

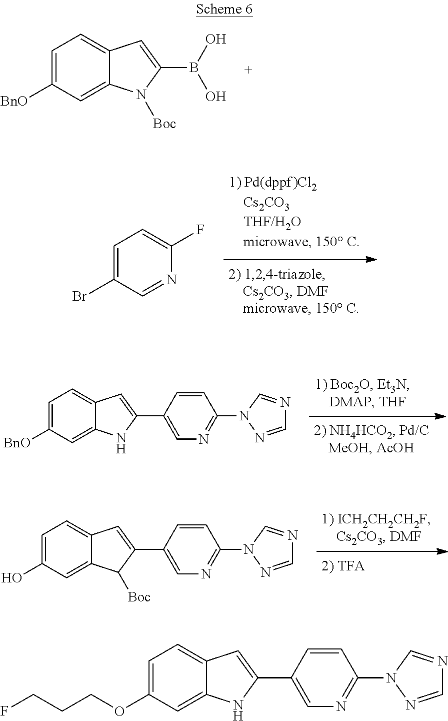

### EXAMPLE 6

### 6-(3-fluoropropoxy)-2-[6-(1H-1,2,4-triazol-1-yl)pyridin-3-yl]-1H-indole

### Step 1: 6-Benzyloxy-2-(6-fluoro-pyridin-3-yl)-indole-1-carboxylic acid tent-butyl ester

A mixture containing 6-benzyloxy-indole-1-Boc-2-boronic acid (1 g, 2.72 mmol), 5-bromo-2-fluoropyridine (484 mg, 2.75 mmol), Pd(dppf)Cl₂.CH₂Cl₂ (56 mg, 0.068 mmol), 1 M aqueous Cs₂CO₃ (2.72 mL, 2.72 mmol), and THF (11 mL) was microwaved to 150° C. for 15 minutes in a sealed microwave tube. After cooling to room temperature, the aqueous layer was removed by pipette and the remaining organics were filtered then concentrated affording a crude residue containing 6-Benzloxy-2-(6-fluoro-pyridin-3-yl)-indole-1-carboxylic acid tert-butyl ester. ES MS (M+H⁺)=419.

### Step 2: 6-benzyloxy-2-(6-[1,2,4]triazol-1-yl-pyridin-3-yl)-1H-indole

To a solution of the crude residue from Step 1 in DMF (10 mL) were added 1,2,4-triazole (376 mg, 5.44 mmol) and Cs₂CO₃ (886 mg, 2.72 mmol). The resulting mixture was heated by microwave to 150° C. for 20 min. After cooling, the resulting mixture was filtered, concentrated and partitioned in water/EtOAc. The organics were collected, dried, filtered and concentrated to afford a crude residue, which was crystallized from CH₂Cl₂ to afford 6-benzyloxy-2-(6-[1,2,4]triazol-1-yl-pyridin-3-yl)-1H-indole (430 mg, 1.17 mmol, 43% yield over two steps) which was used without further purification. ES MS (M+H⁺)=368.

### Step 3: 6-Benzyloxy-2-(6-[1,2,4]triazol-1-yl-pyridin-3-yl)-indole-1-carboxylic acid tert-butyl ester

To a solution of 6-benzyloxy-2-(6-[1,2,4]triazol-1-yl-pyridin-3-yl)-1H-indole (380 mg, 1.034 mmol) in THE (5 mL) was added Boc₂O (0.266 mL, 1.24 mmol), Et₃N (0.173 mL, 1.24 mmol), and DMAP (13 mg, 0.1 mmol) and the resulting mixture was stirred at room temperature for 2 h. Water was then added (10 mL), and the resulting mixture was extracted with EtOAc. The combined organics were dried (Na₂SO₄), filtered, and concentrated to afford a crude mixture containing 6-Benzyloxy-2-(6-[1,2,4]triazol-1-yl-pyridin-3-yl)-indole-1-carboxylic acid tert-butyl ester which was used in subsequent steps without further purification. ES MS (M+H⁺)=468.

### Step 4: 6-Hydroxy-2-(6-[1,2,4]triazol-1-yl-pyridin-3-yl)-indole-1-carboxylic acid tert-butyl ester

To a solution of crude 6-benzyloxy-2-(6-[1,2,4]triazol-1-yl-pyridin-3-yl)-1H-indole from the previous step in 10:1 MeOH/AcOH (10 mL) was added Pd/C (1.1 g, 1.0 mmol) and ammonium formate (630 mg, 10 mmol). After stirring at ambient temperature for 2 h, the resulting mixture was filtered through celite and concentrated in vacuo affording 6-Hydroxy-2-(6-[1,2,4]triazol-1-yl-pyridin-3-yl)-indole-1-carboxylic acid tert-butyl ester (270 mg, 0.715 mmol, 72% yield). ES MS (M+H⁺)=378.

### Step 5: 6-(3-fluoropropoxy)-2-[6-(1H-1,2,4-triazol-1-yl)pyridin-3-yl]-1H-indole

To a solution of 6-Hydroxy-2-(6-[1,2,4]triazol-1-yl-pyridin-3-yl)-indole-1-carboxylic acid tert-butyl ester (30 mg, 0.079 mmol) in DMF (1 mL) was added Cs₂CO₃ (78 mg, 0.24 mmol) and 3-fluoro-1-iodopropane (15 mg, 0.079 mmol). The resulting mixture was stirred at ambient temperature for 12 h, then filtered and concentrated leaving a crude residue that was dissolved in TFA (1 mL) and stirred for 2 h. Concentration of this mixture, provided a crude residue that was purified by reversed phase HPLC to afford 6-(3-fluoropropoxy)-2-[6-(1H-1,2,4-triazol-1-yl)pyridin-3-yl]-1H-indole (23.5 mg, 0.070 mmol, 88% yield). ES MS (M+H⁺)=338; ¹H NMR (499 MHz, DMSO): δ 11.60 (1H, s), 9.40 (1H, s), 8.99 (1H, d, J=2.32 Hz), 8.43 (1H, dd, J=8.56, 2.38 Hz), 8.33 (1H, s), 7.94 (1H, d, J=8.55 Hz), 7.47 (1H, d, J=8.62 Hz), 7.05 (1H, s), 6.93 (1H, d, J=2.14 Hz), 6.73 (1H, dd, J=8.61, 2.24 Hz), 4.73-4.58 (2H, m), 4.12 (2H, t, J=6.25 Hz), 2.21-2.09 (2 H, m); HRMS m/z 338.1417 (C₁₈H₁₆FN₅O+H⁺ requires 338.1412).

### BIOLOGICAL EXAMPLES

Homogenates from AD and non-AD human brain samples were assessed for their immunoreactivity to anti-Aβ antibody 6E10. The highest and lowest levels of 6E10 immunoreactivity were chosen for the AD group and the non-AD control group, respectively. Candidate Aβ compounds were initially selected based on their structural similarity to published amyloid ligands and then for high affinity in competing with [³H]PIB binding to AD brain homogenates. These compounds were radiolabeled with [³H] and tested for binding affinity to human AD brain homogenates as well as binding to human non-AD brain homogenates. [³H]-DMAB (see structure below) was selected based from these candidates based on its binding affinity for human AD brain homogenates, and minimal binding to non-AD control homogenates. A low fraction of non-displaceable binding was also an important criterion.

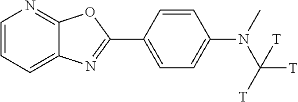

### Structure of [³H]-DMAB (T=tritium)

PET radiotracer candidate compounds were then selected based on their high affinity competition with [³H]-DMAB in binding to AD brain homogenates. These PET radiotracer candidate compounds were tested to determine if they were effective PgP substrates. Those PET radiotracer candidate compounds with little PgP substrate activity were radiolabeled with [³H] or [¹⁸F] and tested for binding affinity to human AD brain homogenates as well as binding to human non-AD brain homogenates and in autoradiographic studies using human AD and non-AD brain slices. Candidate radioligands were selected based on their strong binding affinity for human AD brain homogenates, and minimal binding to non-AD control homogenates. A low fraction of non-displaceable binding was also an important criterion. Minimization of white matter binding was an important criterion.

### Tissue Homogenate Binding Assay:

Postmortem frozen human brain samples from donors with clinical diagnosis of Alzheimer's diseases (AD) or normal control subjects (non-AD) were purchased from Analytical Biological Services Inc., at 701-4 Cornell Business Park, Wilmington, Del. 19801. Brain homogenates of frontal cortex were prepared, divided into aliquots and stored at −70° C. prior to use.

[³H]-DMAB was synthesized at a specific activity of ˜80 Ci/mmol. The final concentration of radioligand for tissue homogenate binding assay was 1.5 nM. Brain homogenates were diluted with phosphate buffered saline (PBS) to 0.4 mg/mL from original 10 mg/mL volume and 200 μl was used in assay for a final concentration of 50 μg/assay tube. Unlabeled test compounds were dissolved in dimethylsulfoxide (DMSO) at 1 mM. Dilution of test compound to various concentrations was made with PBS containing 2% DMSO. Total binding was defined in the absence of competing compound, and non-displaceable binding was determined in the presence of 1 μM unlabeled self block. Compound dilutions (10×) were added into the assay tube (25 μL each/per tube, separately) containing 200 μL brain homogenate dilution, and the tubes were pre-incubated at room temperature for 10 minutes. Then radioligand dilutions (10×) were added into the assay tube (25 μL each/per tube, separately) to a final volume of 250 μl per tube. Incubation was carried out at room temperature (25° C.) for 90 minutes, and then the assay samples were filtered onto GF/C filters using Skatron 12 well harvester, washing on setting 5−5−5 ((˜3×2 ml) ice cold buffer (PBS, pH 7.4). GF/C filter papers for the Skatron harvester were pre-soaked in 0.1% BSA for 1 hour at room temperature before use. Filters were punched into scintillation vials and counted in 2 mL Ultima Gold on Perkin Elmer Tri-Carb 2900TR for 1 minute. The data analysis was done with Prism software. All assays were done in triplicate, and in the laboratory designated for studies using human tissues.

### In Vitro Autoradiography:

Postmortem frozen human brain samples from donors with clinical diagnosis of Alzheimer's diseases (AD) or natural control subjects (non-AD) were purchased from a commercial source. Frozen brain slices (20 μm thickness) were prepared using a cryostat (Leica CM3050) and kept in sequential order. The tissue slices were placed on Superfrost Plus glass slides (Cat. #5075-FR, Brain Research Laboratories, USA), dried at room temperature, and stored in a slide box at −70° C. before use. The final concentration of radioligand for in vitro autoradiography was 1.0 nM. On the day of a binding experiment, adjacent slices were selected from each brain region of interest for in vitro autoradiographic study, and were designated as total binding and non-specific binding (NSB). These slices were thawed at room temperature for 15 minutes in a biosafety hood. Total binding of radioligand in a brain slices was defined in the absence of competitor, and non-specific binding (NSB) was determined in the presence of competitor (1.0M unlabeled compound). The brain slides were first pre-incubated at room temperature for twenty minutes in PBS buffer, pH 7.4. The slices were then transferred to fresh buffer containing radioligand or radioligand plus competitor as described above, and incubated at room temperature for ninety minutes. Incubation was terminated by washing the slices three times in ice cold (4° C.) wash buffer (PBS, pH 7.4) with each wash lasting three minutes. After washing, the slices were briefly rinsed in ice cold (4° C.) deionized water, and then dried completely by an air blower at room temperature. The slices were placed against Fuji Phosphor Image Plates (TR25, Fuji) in a sealed cassette for exposure at room temperature. After one week exposure, the plates were scanned in Fuji BAS 5000 Scanner, and the scanned images were analyzed using MCID 7.0 software. [³H]-microscales (Amersham Biosciences, GE), were used for quantification of radioligand binding density. All the slice binding assays were done in the laboratory designated for studies using human tissues.

Candidate radioligands that fit these criteria were radiolabeled with [¹⁸F]. The [¹⁸F] labeled radioligands were characterized in vivo in rhesus monkey for rapid uptake into and clearance from brain. In selecting the final PET radiotracer, minimization of retention in white matter was an important criterion.

### Assessment of Amyloid Load:

Subjects are administered a Mini-Mental State Examination to assess whether they are normal control subjects or are AD patients. PET studies are performed on both groups of patients using the PET radiotracers described herein, and using methods known to those versed in the art. Uptake and retention of radiotracer in regions where amyloid plaque is known to accumulate (e.g., frontal cortical regions) is compared with uptake and retention of radiotracer in a reference region where amyloid plaque does not accumulate (e.g., cerebellum). The difference in uptake and retention between these pairs of regions is greater for the AD patients compared to the normal control subjects; this greater difference is due to the greater AP plaque load in the AD patients. Test-retest (intra-subject) variability is established by a second, essentially identical PET study.

To determine if a compound is effective for reducing amyloid plaque, a PET study is performed prior to administering the plaque reducing compound. After a course of treatment with the therapeutic compound, a second PET study is performed. A reduction in uptake and retention of the PET radiotracer in the regions in which plaque is known to accumulate (greater than the test-retest variability) indicates a reduction in the plaque load. In such a study each subject serves as his or her own pretreatment control.

The compounds of this invention possess IC50 values in the human AD brain tissue homogenate assay in the range of 0.1 nM-1000 nM. For example, the IC50 of the following compounds are:

| Compound | IC50 in Tissue Homogenate Assay |
|---|---|
| 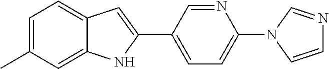 | 245 nM |
| 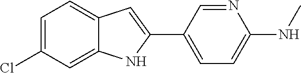 | 40 nM |
| 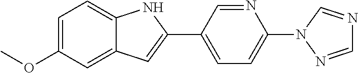 | 10 nM |
| 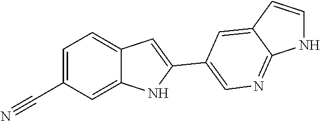 | 79 nM |

1-23. (canceled)

24. A compound represented by Formula I: 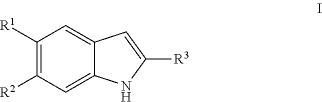
or a pharmaceutically acceptable salt, solvate or in vivo hydrolysable ester thereof, wherein:
R³ is pyridyl optionally substituted with 1 to 3 groups of R⁴, R⁵, or R⁶, with the proviso that when two of R⁴, R⁵ and R⁶, is hydrogen and the remainder of R⁴, R⁵ or R⁶ is N(R²)₂, piperazinyl or methyl piperazinyl, and one of R¹ and R² is hydrogen then the other of R¹ and R² is not methoxy or halogen;
R represents hydrogen, or —C₁₋₆alkyl, said alkyl optionally substituted with halo;
R¹, R², R⁴, R⁵, and R⁶ independently represent hydrogen, —C₅₋₁₀ aryl, —C₅₋₁₀ heterocyclyl, —N(R²)₂, CN, —(CH₂)ₙhalo, CF₃, —O(CH₂)ₙR, —O(CH₂)ₙC₅₋₁₀ heterocyclyl, —C₁₋₆alkyl, —OCF₃, —O(CH₂)ₛF, —(O(CH₂)ₛ)ₚ(CH₂)ₛhalo, —(O(CH₂)ₛ)ₚOR, said alkyl, aryl, and heterocyclyl optionally substituted with 1 to 3 groups of R^{a}, or when two of R⁴, R⁵ and R⁶ are adjacent to each other on the R³ pyridyl then they may combine with the atoms to which they are attached to form a 9-10 membered heterocyclic ring optionally interrupted by NR, O, or S, said heterocyclyl optionally substituted with 1 to 3 groups of R^{a};
R^{a} represents —CN, NO₂, halo, CF₃, —C₁₋₆alkyl, —C₁₋₆alkenyl, —C₁₋₆alkynyl, —(CH₂)ₙhalo, —OR, —NRR¹, —C(═NR¹)NR²R⁵,—NR¹COR², —NR¹CO₂R², —NR¹SO₂R⁵, —NR¹CONR²R⁵,—SR⁵, —SOR⁵, —SO₂R⁵, —SO₂NR¹R², —COR¹, —CO₂R¹, —CONR¹R², —C(═NR¹)R², or —C(═NOR¹)R²;
n represents 0-6;
s represents 1-6; and
p represents 1-3.

25. The compound according to claim 24 wherein R³ is substituted with halo, methylamine, piperazinyl, triazolyl, imidazolyl, or pyrazolyl.

26. The compound according to claim 24 wherein R³ is pyridyl substituted with florine, triazolyl, or imidazolyl.

27. The compound according to claim 24 wherein when two of R⁴, R⁵ and R⁶ adjacent to each other on the R³ pyridyl combine with the atoms to which they are attached to form a 9-10 membered heterocyclic ring including fused rings, optionally interrupted by NR, O, or S, said heterocyclic ring optionally substituted with R^{a}.

28. The compound according to claim 27 represented by structural formula II: 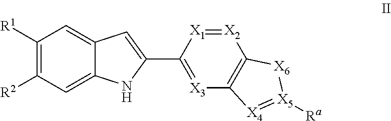
or a pharmaceutically acceptable salt, solvate or in vivo hydrolysable ester thereof, wherein:
X₁-X₅ are Nor CH, provided only one of X₁-X₃ is N at any given time; and X₆ is NR, —O—, CH₂ or S and all other variables are as previously described.

29. The compound according to claim 28 wherein X₁ through X₆ form a pyrrolo pyridinyl and all other variables are as previously described.

30. The compound according to claim 24 wherein R¹ and R² are selected from the group consisting of hydrogen, CN, —(CH₂)ₙhalo, —O(CH₂)ₙR, —O(CH₂)ₙhalo, —O(CH₂)ₙC₅₋₁₀ heterocyclyl, O(CH₂)ₙC₆₋₁₀ aryl or —C₁₋₆alkyl.

31. The compound according to claim 30 wherein one of R¹ and R² is hydrogen and the other is O(CH₂)ₙF, F, Br, Cl, CN, methoxy, methyl, hydroxyl, benzyloxy.

32. The compound according to claim 24 wherein the compounds of formula I are isotopically labeled with ²H, ³H, ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁶Br, ⁷⁷Br, ¹²³I, ¹²⁴I or ¹³¹I.

33. A compound which is:
6-(benzyloxy)-2-[6-(1H-imidazol-1-yl)pyridin-3-yl]-1H-indole,
6-chloro-2-[6-(1H-imidazol-1-yl)pyridin-3-yl]-1H-indole,
2-[6-(1H-imidazol-1-yl)pyridin-3-yl]-6-methyl-1H-indole,
2-[6-(1H-imidazol-1-yl)pyridin-3-yl]-6-methoxy-1H-indole,
2-[6-(1H-imidazol-1-yl)pyridin-3-yl]-1H-indole-6-carbonitrile,
5-fluoro-2-[6-(1H-imidazol-1-yl)pyridin-3-yl]-1H-indole,
2-[6-(1H-imidazol-1-yl)pyridin-3 -yl]-5-methyl-1H-indole,
2-[6-(1H-imidazol-1-yl)pyridin-3-yl]-1H-indol-5-ol,
2-[6-(1H-imidazol-1-yl)pyridin-3-yl]-5-methoxy-1H-indole,
5-bromo-2-[6-(1H-imidazol-1-yl)pyridin-3-yl]-1H-indole,
2-[6-(1H-imidazol-1-yl)pyridin-3-yl]-1H-indole-5-carbonitrile,
5-(6-chloro-1H-indol-2-yl)-N-methylpyridin-2-amine,
N-methyl-5-(6-methyl-1H-indol-2-yl)pyridin-2-amine,
5-(6-methoxy-1H-indol-2-yl)-N-methylpyridin-2-amine,
2-[6-(methylamino)pyridin-3-yl]-1H-indole-6-carbonitrile,
5-(5-fluoro-1H-indol-2-yl)-N-methylpyridin-2-amine,
N-methyl-5-(5-methyl-1H-indol-2-yl)pyridin-2-amine,
5-(5-methoxy-1H-indol-2-yl)-N-methylpyridin-2-amine,
5-(5-bromo-1H-indol-2-yl)-N-methylpyridin-2-amine,
2-[6-(methylamino)pyridin-3-yl]-1H-indole-5-carbonitrile,
6-(benzyloxy)-2-(6-fluoropyridin-3-yl)-1H-indole,
6-chloro-2-(6-fluoropyridin-3-yl)-1H-indole,
2-(6-fluoropyridin-3-yl)-6-methyl-1H-indole,
2-(6-fluoropyridin-3-yl)-6-methoxy-1H-indole,
2-(6-fluoropyridin-3-yl)-1H-indole-6-carbonitrile,
5-fluoro-2-(6-fluoropyridin-3-yl)-1H-indole,
2-(6-fluoropyridin-3-yl)-5-methyl-1H-indole,
2-(6-fluoropyridin-3-yl)-1H-indol-5-ol,
2-(6-fluoropyridin-3-yl)-5-methoxy-1H-indole,
5-bromo-2-(6-fluoropyridin-3-yl)-1H-indole,
2-(6-fluoropyridin-3-yl)-1H-indole-5-carbonitrile,
6-(benzyloxy)-2-[6-(1H-1,2,4-triazol-1-yl)pyridin-3-yl]-1H-indole,
6-chloro-2-[6-(1H-1,2,4-triazol-1-yl)pyridin-3-yl]-1H-indole,
6-methyl-2-[6-(1H-1,2,4-triazol-1-yl)pyridin-3-yl]-1H-indole,
6-methoxy-2-[6-(1H-1,2,4-triazol-1-yl)pyridin-3-yl]-1H-indole,
2-[6-(1H-1,2,4-triazol-1-yl)pyridin-3-yl]-1H-indole-6-carbonitrile,
5-fluoro-2-[6-(1H-1,2,4-triazol-1-yl)pyridin-3-yl]-1H-indole,
5-methyl-2-[6-(1H-1,2,4-triazol-1-yl)pyridin-3-yl]-1H-indole,
5-methoxy-2-[6-(1H-1,2,4-triazol-1-yl)pyridin-3-yl]-1H-indole,
5-bromo-2-[6-(1H-1,2,4-triazol-1-yl)pyridin-3-yl]-1H-indole,
6-(benzyloxy)-2-[6-(4-methylpiperazin-1-yl)pyridin-3-yl]-1H-indole,
6-chloro-2-[6-(4-methylpiperazin-1-yl)pyridin-3-yl]-1H-indole,
6-methyl-2-[6-(4-methylpiperazin-1-yl)pyridin-3 -yl]-1H-indole,
6-methoxy-2-[6-(4-methylpiperazin-1-yl)pyridin-3-yl]-1H-indole,
2-[6-(4-methylpiperazin-1-yl)pyridin-3-yl]-1H-indole-6-carbonitrile,
5-fluoro-2-[6-(4-methylpiperazin-1-yl)pyridin-3-yl]-1H-indole,
5-methyl-2-[6-(4-methylpiperazin-1-yl)pyridin-3-yl]-1H-indole,
2-[6-(4-methylpiperazin-1-yl)pyridin-3-yl]-1H-indol-5-ol,
5-methoxy-2-[6-(4-methylpiperazin-1-yl)pyridin-3-yl]-1H-indole,
5-bromo-2-[6-(4-methylpiperazin-1-yl)pyridin-3-yl]-1H-indole,
2-[6-(4-methylpiperazin-1-yl)pyridin-3-yl]-1H-indole-5-carbonitrile,
6-(3-fluoropropoxy)-2-[6-(1H-1,2,4-triazol-1-yl)pyridin-3-yl]-1H-indole,
5-(3-fluoropropoxy)-2-[6-(1H-1,2,4-triazol-1-yl)pyridin-3-yl]-1H-indole,
6-(2-fluoroethoxy)-2-[6-(1H-1,2,4-triazol-1-yl)pyridin-3-yl]-1H-indole,
5-(2-fluoroethoxy)-2-[6-(1H-1,2,4-triazol-1-yl)pyridin-3-yl]-1H-indole,
5-(6-methyl-1H-indol-2-yl)-1H-pyrrolo[2,3-b]pyridine,
2-(1H-pyrrolo[2,3-b]pyridin-5-yl)-1H-indole-6-carbonitrile,
5-(5-methoxy-1H-indol-2-yl)-1H-pyrrolo[2,3-b]pyridine,
5-(5-bromo-1H-indol-2-yl)-1H-pyrrolo[2,3-b]pyridine,
2-(1H-pyrrolo[2,3-b]pyridin-5-yl)-1H-indole-5-carbonitrile,
5-(6-methoxy-1H-indol-2-yl)-1H-pyrrolo[2,3-b]pyridine,
5-(5-methyl-1H-indol-2-yl)-1H-pyrrolo[2,3-b]pyridine,
5-(6-chloro-1H-indol-2-yl)-1H-pyrrolo[2,3-b]pyridine,
or a pharmaceutically acceptable salt, solvate or in vivo hydrolysable ester thereof.

34. The compound according to claim 34 which isotopically labeled as ¹¹C, ¹³C, ¹⁴C, ¹⁸F, ¹⁵O, ¹³N, ³⁵S, ²H, or ³H.

35. The compound according to claim 34 which is
6-(3-fluoropropoxy)-2-[6-(1H-1,2,4-triazol-1-yl)pyridin-3-yl]-1H-indole,
5-(3-fluoropropoxy)-2-[6-(1H-1,2,4-triazol-1-yl)pyridin-3-yl]-1H-indole,
6-(2-fluoroethoxy)-2-[6-(1H-1,2,4-triazol-1-yl)pyridin-3-yl]-1H-indole,
5-(2-fluoroethoxy)-2-[6-(1H-1,2,4-triazol-1 -yl)pyridin-3 -yl]-1H-indole, or a pharmaceutically acceptable salt, solvate or in vivo hydrolysable ester thereof.

36. A pharmaceutical composition comprising a compound according to claim 24 and a pharmaceutically acceptable carrier.

37. A composition for imaging of amyloid deposits, comprising a radio-labeled compound of claim 24 and a pharmaceutically acceptable carrier.

38. A method of inhibiting amyloid plaque aggregation in a mammal, or for measuring amyloid deposits in a patient comprising administering the composition of claim 37 in an amount effective to inhibit amyloid plaque aggregation.

39. The method according to claim 38 wherein detection is carried out by performing positron emission tomography (PET) imaging, single photon emission computed tomography (SPECT), magnetic resonance imaging, or autoradiography.

40. A method for preventing and/or treating or for diagnosing and monitoring the treatment of Alzhemier's Disease, familial Alzheimer's Disease, Cognitive Deficit in Schizophrenia, Down's Syndrome and homozygotes for the apolipoprotein E4 allele comprising administering to a patient in need thereof a therapeutically effective amount of a compound according to claim 24.
